(19) United States
(12) Patent Application Publication
(10) Pub. No.: US 2014/0316710 A1
(43) Pub. Date: Oct. 23, 2014

(54) METHOD AND APPLICATION FOR DETERMINING AND VISUALLY DEPICTING THE PASSING PROBABILITY OF A MARIJUANA/CANNABIS DRUG TEST

(71) Applicant: Jason Mahoney, Wichita, KS (US)

(72) Inventor: Jason Mahoney, Wichita, KS (US)

(21) Appl. No.: 13/866,015

(22) Filed: Apr. 18, 2013

(51) Int. Cl. G01N 33/50

(52) U.S. Cl. CPC G01N 33/50
USPC 702/19

(57) ABSTRACT

Systems, methods and apparatus are provided through which in some implementations the clinical detectability of THC exposure is accurately estimated.

### FIELD

The field of the invention is substance testing.

### BACKGROUND

Conventional systems, methods and apparatus provide estimates of time that transpires before a person will pass a Tetrahydrocannabinol (THC) or THC metabolite test. The estimates of time are based on trivial calculations that are highly inaccurate. THC is the primary psychoactive compound of the Marijuana/Cannabis plant and THC and metabolites thereof are used to verify use of the Marijuana/Cannabis plant.

### BRIEF DESCRIPTION

In one aspect, an electronic device includes a processor, a display device and a memory operable to store processor instructions to receive at least one representation of characteristics of the person, receive at least one representation of the marijuana use of the person, determine the likelihood that the person will pass a drug test a certain period of time after cessation of intake of THC, the likelihood represented as a percentage, and render on the display device a visual presentation of the percentage of the likelihood.

In a further aspect, a non-transitory machine readable medium storing a program for enabling a computer to perform a method that includes receiving representations of characteristics of the person, receiving representations of the marijuana use of the person, determining the likelihood that the person will pass a drug test a certain period of time after cessation of intake of THC, the likelihood represented as a percentage, and displaying a visual presentation of the percentage of the likelihood.

In another aspect, a method includes receiving representations of characteristics of a person, receiving representations of the marijuana/cannabis use of the person, determining the likelihood that the person will pass a drug test a certain period of time after cessation of intake of THC, the likelihood represented as a percentage and displaying a visual presentation of the percentage of the likelihood.

In yet a further aspect, software reports probability of passing a THC or THC metabolite test after a given period of time after taking as inputs data pertaining to the user and their use of marijuana or other substances.

In yet an additional aspect, software outputs probabilities of passing a THC or THC metabolite test.

In a further additional aspect, statistical models are generated to predict probabilities of passing a THC or THC metabolite test.

Aspects of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 is a flowchart of a method of drug testing, according to an implementation;

FIG. 2 is a block diagram of an apparatus of drug testing, according to an implementation;

FIG. 3 illustrates a graph of the percentage of the likelihood, according to an implementation;

FIG. 4 is a block diagram of a hardware and operating environment in which different implementations can be practiced; and

FIG. 5 is a block diagram of a mobile device, according to an implementation.

### DETAILED DESCRIPTION

In the following detailed description, reference is made to the accompanying drawings that form a part hereof, and in which is shown by way of illustration specific implementations that may be practiced. These implementations are described in sufficient detail to enable those skilled in the art to practice the implementations, and it is to be understood that other implementations may be utilized and that logical, mechanical, electrical and other changes may be made without departing from the scope of the implementations. The following detailed description is, therefore, not to be taken in a limiting sense.

The detailed description is divided into four sections. In the first section, method described. In the second section, apparatus is described. In the third section, hardware and operating environments in conjunction with which implementations may be practiced are described. The fourth section, a conclusion of the detailed description is provided.

### Method

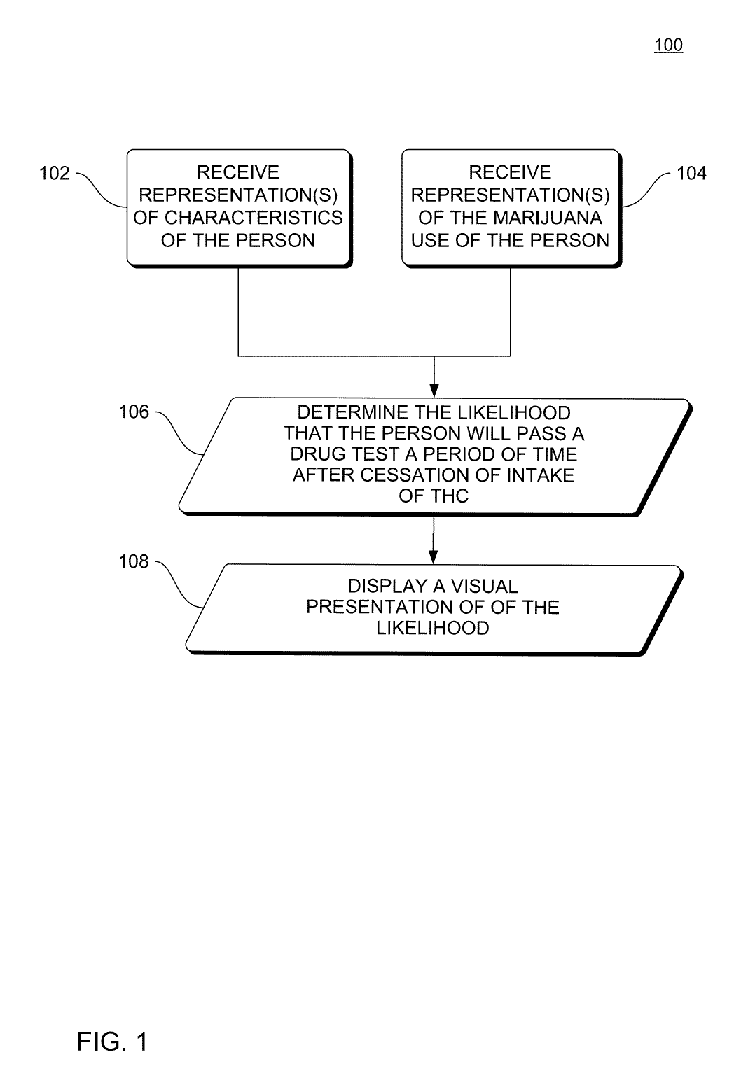

FIG. 1 is a flowchart of a method 100 of drug testing, according to an implementation.

Method 100 includes receiving representations of characteristics of the person, at block 102. In some implementations, a user submits their measurements through a website, the characteristics include gender, age, height, weight, and waist size. In some implementations, a user submits, on a scale from 1 (lowest, shortest, slowest) to 5 (highest, longest, fastest) marijuana smoking frequency, average marijuana potency, average amount smoked, estimated metabolic speed, cardio activity level, and muscular composition level. In some implementations, the user submits a reason why the user was tested (random, pre-employment, etc.) and a representation of the ethnicity of the user.

Method 100 also includes receiving representations of the marijuana or cannabis use of the person, at block 104. In some implementations, the user submits the urine drug test results through a website. In some implementations, the user submits urine, hair, saliva, or blood test results through a website. In some implementations, the user submits identification of how many non-smoking days before taking the urine test, whether or not the user attempted diluting or detoxing their urine, whether the urine was tested at home/on site or by a lab, and in some situations most importantly, if the user passed their urine test or not. Urine dilution is a process in which a person consumes large amounts of fluids (water, tea, cranberry juice, etc.) a few hours before providing a urine sample in an attempt to lower the concentration levels of THC or THC metabolites in their urine. Urine detoxification occurs when a person consumes a product that has claims relating to: removing impurities, cleaning a person's body, flushing a person's system, or (more bluntly) passing a drug test. Most detoxification products instruct their users to consume large amount of fluids (similar to urine dilution) after or during the consumption of the detoxifying product. However, the passing percentages of urine detoxification yield the same results as urine dilution. Both of these methods have the potential to influence validity markers used by laboratories in order to verify the validity of a urine specimen. Validity markers include: pH levels, creatinine levels, nitrite levels, specific gravity, and creatinine/THC or THC metabolite ratios. Laboratories can choose to reject a urine specimen on grounds of dilution and request another urine sample or outright fail the urine sample if any of the validity markers are out of normal range.

Method 100 also includes determining the likelihood that the person will pass a drug test after a certain period of time of cessation of intake of the THC, the likelihood represented as a percentage, at block 106. Urine and Hair drug tests use THC metabolites to determine the likelihood or a pass/fail result. Blood and Saliva testing uses THC (not THC metabolites) to determine the likelihood or the pass/fail result.

Method 100 also includes displaying a visual presentation of the percentage of the likelihood, at block 108.

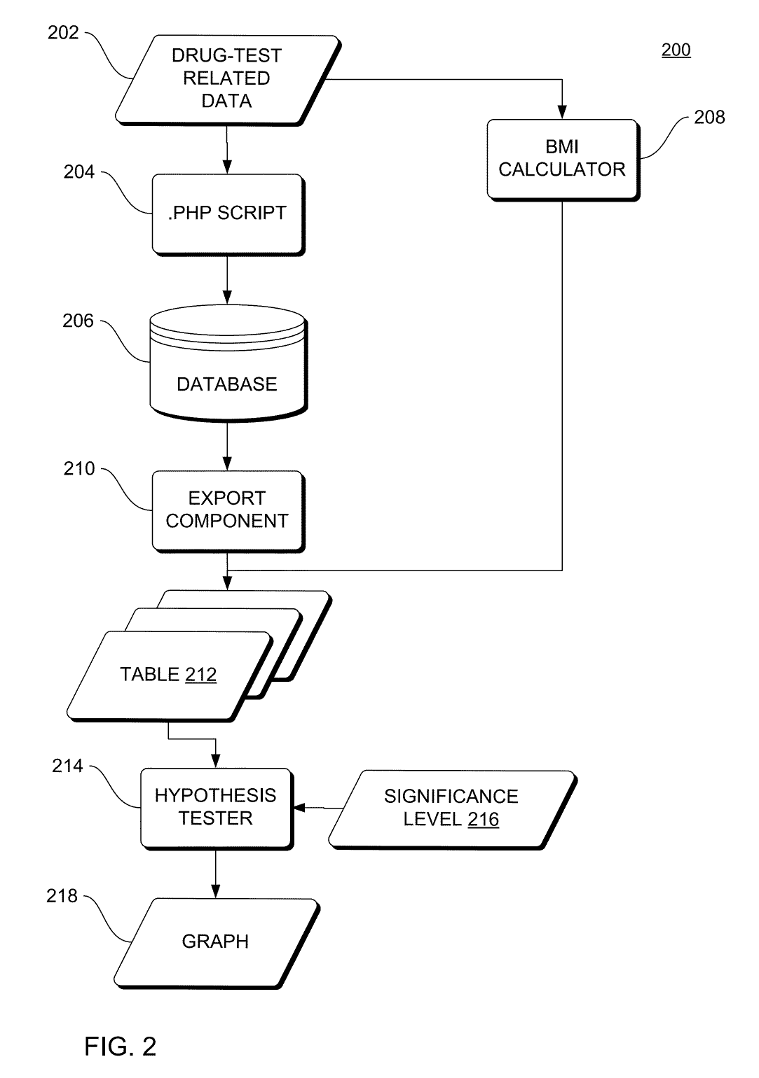

FIG. 2 is a block diagram of an apparatus 200 of drug testing, according to an implementation.

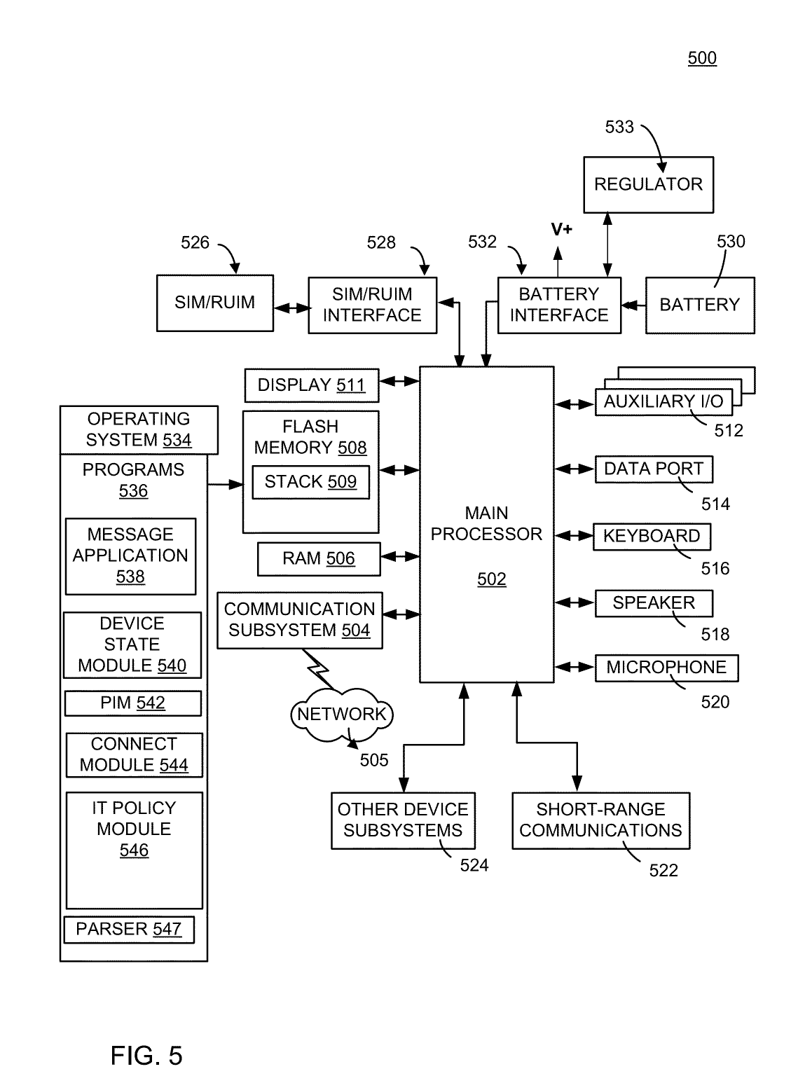

Apparatus 200 includes a .php script 204 that handles drug-test related data 202 that is received in steps 102 and 104 of the method 100 in FIG. 1. The drug-test related data 202 includes the representations of characteristics of the person and the representations of the marijuana or cannabis use of the person. The .php script 204 also inserts and updates the data 202 into a MySQL database 206. In some implementations, the .php script 204 uses the IP Address or appropriate identification marker of the electronic device of the user as the primary key. One example of the electronic device is mobile device 500 in FIG. 5 that operates the Android® operating system or the Apple® iOS operating system. When the data 202 is submitted, a check is performed to determine if the IP Address or appropriate identification marker exists in the database 206. If the IP address or appropriate identification marker does not exist in the database 206, all of the received data 202 is inserted into a table of the database 206. If the IP Address or appropriate identification marker exists in the database 206, only necessary changes are updated in the table of the database 206. The purpose of updating data related to the IP address or appropriate identification marker in the database 206 is to prevent a single user from submitting multiple datasets of data 202 and as a result, negatively impacting the predictions that are made from the aggregated data in the database 206. Updating the data related to the IP address or appropriate identification marker in the database 206 also helps prevent malicious attacks if someone were to purposely submit multiple datasets from a single location. The .php script 204 also handles MySQL Injection Attacks.

In some implementations, a body mass index (BMI) calculator 208 determines BMI, body fat percent of the user, and weight of the user's body fat from either the mass of the user, waist size of the user, and/or the height of the user in inches and pounds in the drug-test related data 202 using one of the following formulas:

BMI=mass (lbs)/((height (in))̂2)*703 Body Mass Index Formula 1

Men: BF %=((4.15*waist−0.082*weight−98.42)/weight)*100

Women: BF %=((4.15*waist−0.082*weight−76.76)/weight)*100 Body Fat Percent Formulas 1 & 2 (YMCA Method)

Weight of BF=BF %*weight Weight of Body Fat Formula 1

Some implementations do not include the BMI Calculator 208, in which case, the .php script 204 calculates BMI, BF %, and BF Weight immediately after the user submits their data and correctly inserts/updates these values into the database 206.

An exporter component 210 exports each data table as an Excel Spreadsheet table 212. In some implementations, datasets are exported tables based on the type of test taken (lab or home/on-site) and whether or not the user detoxed/diluted their urine, which results in 6 tables. Each of the 6 tables are separated by whether or not the users passed the urine test, which results in 12 tables:
Lab Tested (Passed)
Lab Tested with Dilution (Passed)
Lab Tested with Detox (Passed)
Home/On-Site Tested (Passed)
Home/On-Site Tested with Dilution (Passed)
Home/On-Site Tested with Detox (Passed)
Lab Tested (Failed)
Lab Tested with Dilution (Failed)
Lab Tested with Detox (Failed)
Home/On-Site Tested (Failed)
Home/On-Site Tested with Dilution (Failed)
Home/On-Site Tested with Detox (Failed)

Within each of these 12 tables are 13 sub-categories: Age, Height, Body Weight (Lbs), Waist (Inches), BMI, Body Fat Percent, Body Fat Weight (Lbs), Smoking Frequency, Marijuana Potency, Amount Smoked Each Day, Estimated Metabolic Speed, Cardio Activity Level, and Muscular Composition Level. Other metrics based on these categories may be discovered, formulated, and/or used in the future. A Linear Regression test is performed on each one of these categories vs. the Days of Uninterrupted Cessation before Test.

A hypothesis tester 214 receives data from the tables 212 and performs a Wald-Wolfowitz Runs Test in reference to the Days of Uninterrupted Cessation before Test variable in the data 202. The hypothesis tester 214 performs a null hypothesis (HO) and alternative hypothesis (HA). The null hypothesis usually states something along the lines of: “there is no relationship between two measured phenomena” and the alternative hypothesis is usually something that states the opposite, such as: “there is a relationship between two measured phenomena.” The hypothesis tester 214 calculates a p-value which is “the probability of obtaining a test statistic at least as extreme as the one that was actually observed, assuming that the null hypothesis is true.” The p-value is then compared with a pre-determined statistical significance level 216. If the p-value is less than the Significance Level of 0.05, then one would reject the null hypothesis that there is no relationship between two measured phenomena. If the p-value is higher, one would fail to reject the null hypothesis. An example of a null hypothesis is: “there is no relationship between the submitted datasets” which is “the data was submitted by random chance.” In some implementations, a p-value significantly greater than 0.05, which fails to reject that “the data was submitted by random chance,” is beneficial because a positive indicator is not detecting a pattern in the way data was submitted.

The hypothesis tester 214 in some implementations, performs a linear regression test that calculates a p-value and an Adjusted R̂2 value. If the p-value <0.05, the null hypothesis that the previously mentioned sub-category under scrutiny had no effect on time (days) is rejected. In the case of the Smoking Frequency variable (p-value=0.0001), the null hypothesis is rejected. This basically means that the Smoking Frequency variable does have an effect on the time needed in order to pass a THC or THC metabolite test and is therefore, statistically significant (in layman's terms).

Any sub-category that fails to reject the null hypothesis is not used in the prediction model. Of those that do reject the null hypothesis, the Adjusted R̂2 (R Squared) value is used to determine how important the variable in question is when doing a prediction. For example, the Smoking Frequency variable makes up 34% of the prediction while the user's belief in Marijuana Potency only makes up 9% of the prediction. There are several other variables that total up to 100%.

For each statistically significant variable, the data is sorted by the user's input vs. Days of Uninterrupted Cessation before Test and the 90^{th}, 75^{th}, 50^{th}, 25^{th} and 10^{th} percentiles are determined. The antagonistic percentiles of the variable are then merged to form a medium, and then the slope and y-intercept numbers are found. (Please see TABLE 1: Smoking Frequency and TABLE 2: Metabolic Speed for clarification). In some implementations, the 90^{th}, 75^{th}, 50^{th}, 25^{th}, and 10^{th} percentile trendlines are plotted on a graph representing a statistically significant variable vs. Days of Uninterrupted Cessation before Test. When doing this, the Days of Uninterrupted Cessation before Test variable may be comprised of passing results, failing results, or a combination of both.

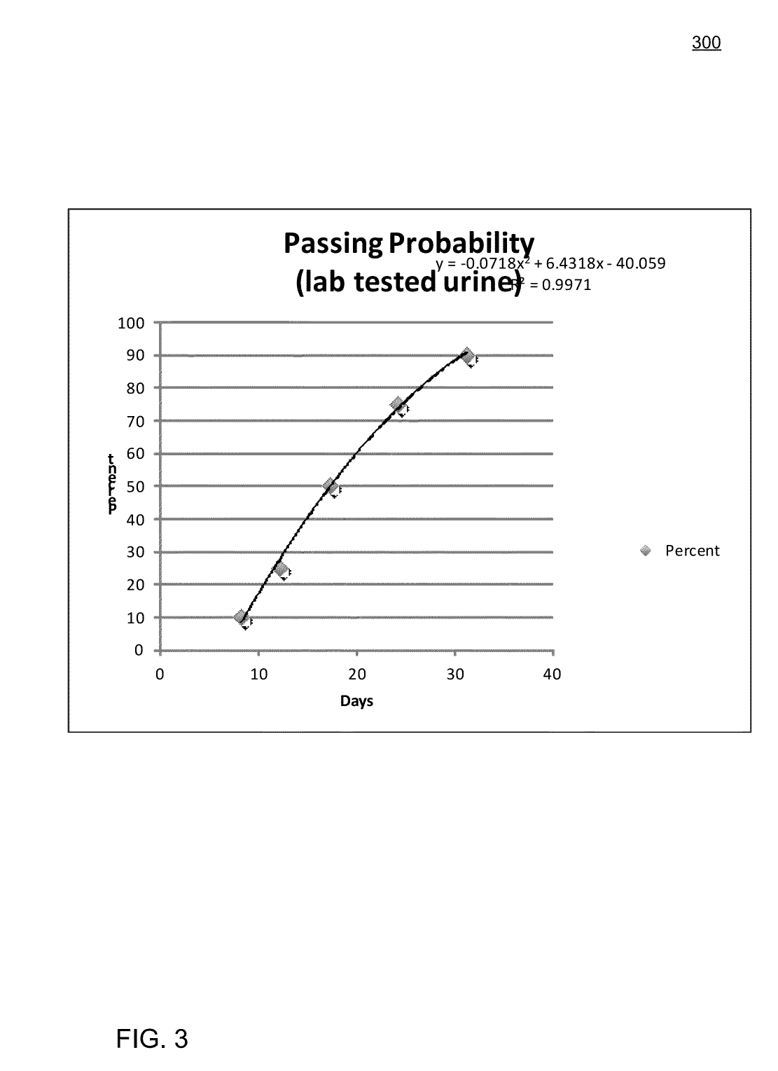

These are all essentially combined at the end (please see TABLE 3: Drug Test Passing Probability Calculations for clarification) to form the Passing Percentile vs. Days data plots 218, which are displayed in action 108 in FIG. 1. The plot displays a passing percentile on the Y-axis vs. time on the X-axis. The graph 218 represents this plot with a polynomial trendline (2nd order). (Please see FIG. 3 for clarification). The trendline equation is located on the top right of the chart. In some implementations, the plot includes six trendlines representing: Lab Tested Urine, Lab Tested Urine with Detox, Lab Tested Urine with Dilution, Home/On-Site Tested Urine, Home/On-Site Tested Urine with Detox, and Home/On-Site Tested Urine with Dilution.

In some implementations, after a person provides a urine sample to an HHS/SAMHSA certified lab, as described in “Current List of Laboratories and Instrumented Initial Testing Facilities Which Meet Minimum Standards To Engage in Urine Drug Testing for Federal Agencies” Federal Register/Vol. 78, No. 42/Monday, Mar. 4, 2013/Notices”, the urine sample is run through an initial test called an immunoassay. For the metabolites of THC, the threshold sensitivity level of 50 ng/mL is the industry standard. If a urine sample is found to have a THC metabolite level that is greater than 50 ng/mL, the sample is flagged as “Suspect—Positive” and tested a second time where the results are confirmed with the use of Gas Chromatography/Mass Spectrometry (GC/MS). The threshold sensitivity level of GC/MS is 15 ng/mL. If the “Suspect—Positive” urine sample has a THC metabolite level that is greater than 15 ng/mL, then the urine sample is confirmed to be positive for marijuana usage. In most cases, the THC metabolite sought for is 11-nor-9-Carboxy-THC, also loosely known as THC-COOH.

In some situations, the immunoassay may have a threshold sensitivity level of 20 ng/mL, which is tracked when a user submits their data to the apparatus 200.

The following table describes an implementation of marijuana smoking frequency in the representations of marijuana/cannabis use of the person:

TABLE 1
Smoking Frequency

| | |
|---|---|
| Test Positive (Fail) | Test Negative (Pass) |

| Frequency | Days | Frequency | Days |
|---|---|---|---|
| 1 | 60 | 1 | 49 |
| 1 | 57 | 1 | 45 |
| 1 | 50 | 1 | 45 |
| 1 | 45 | 1 | 42 |
| 1 | 45 | 1 | 40 |
| 1 | 45 | 1 | 40 |
| 1 | 42 | 1 | 39 |
| 1 | 36 | 1 | 38 |
| 1 | 35 | 1 | 37 |
| 1 | 33 | 1 | 36 |
| 1 | 33 | 1 | 35 |
| 1 | 32 | 1 | 35 |
| 1 | 32 | 1 | 34 |
| 1 | 31 | 1 | 30 |
| 1 | 30 | 1 | 29 |
| 1 | 30 | 1 | 29 |
| 1 | 30 | 1 | 28 |
| 1 | 30 | 1 | 28 |
| 1 | 30 | 1 | 28 |
| 1 | 30 | 1 | 26 |
| 1 | 28 | 1 | 25 |
| 1 | 26 | 1 | 25 |
| 1 | 25 | 1 | 24 |
| 1 | 25 | 1 | 24 |
| 1 | 24 | 1 | 24 |
| 1 | 24 | 1 | 23 |
| 1 | 24 | 1 | 22 |
| 1 | 24 | 1 | 22 |
| 1 | 24 | 1 | 22 |
| 1 | 23 | 1 | 21 |
| 1 | 23 | 1 | 21 |
| 1 | 22 | 1 | 21 |
| 1 | 21 | 1 | 20 |
| 1 | 21 | 1 | 20 |
| 1 | 20 | 1 | 20 |
| 1 | 20 | 1 | 20 |
| 1 | 20 | 1 | 19 |
| 1 | 20 | 1 | 18 |
| 1 | 20 | 1 | 17 |
| 1 | 20 | 1 | 16 |
| 1 | 20 | 1 | 16 |
| 1 | 19 | 1 | 15 |
| 1 | 19 | 1 | 15 |
| 1 | 19 | 1 | 14 |
| 1 | 19 | 1 | 14 |
| 1 | 18 | 1 | 13 |
| 1 | 18 | 1 | 13 |
| 1 | 17 | 1 | 13 |
| 1 | 17 | 1 | 12 |
| 1 | 16 | 1 | 12 |
| 1 | 16 | 1 | 12 |
| 1 | 16 | 1 | 12 |
| 1 | 16 | 1 | 11 |
| 1 | 16 | 1 | 11 |
| 1 | 15 | 1 | 11 |
| 1 | 15 | 1 | 10 |
| 1 | 15 | 1 | 10 |
| 1 | 15 | 1 | 10 |
| 1 | 15 | 1 | 9 |
| 1 | 15 | 1 | 9 |
| 1 | 15 | 1 | 9 |
| 1 | 15 | 1 | 9 |
| 1 | 15 | 1 | 8 |
| 1 | 15 | 1 | 7 |
| 1 | 14 | 1 | 7 |
| 1 | 14 | 1 | 7 |
| 1 | 14 | 1 | 7 |
| 1 | 14 | 1 | 7 |
| 1 | 14 | 1 | 6 |
| 1 | 14 | 1 | 6 |
| 1 | 14 | 1 | 6 |
| 1 | 14 | 1 | 6 |
| 1 | 14 | 1 | 5 |
| 1 | 14 | 1 | 5 |
| 1 | 14 | 1 | 3 |
| 1 | 13 | 1 | 3 |
| 1 | 13 | 1 | 3 |
| 1 | 13 | 1 | 3 |
| 1 | 13 | 1 | 3 |
| 1 | 12 | 1 | 3 |
| 1 | 12 | 1 | 2 |
| 1 | 12 | 1 | 2 |
| 1 | 12 | 1 | 2 |
| 1 | 12 | 1 | 1 |
| 1 | 12 | 1 | 1 |
| 1 | 12 | 2 | 85 |
| 1 | 11 | 2 | 41 |
| 1 | 11 | 2 | 40 |
| 1 | 10 | 2 | 38 |
| 1 | 10 | 2 | 35 |
| 1 | 10 | 2 | 34 |
| 1 | 10 | 2 | 32 |
| 1 | 10 | 2 | 30 |
| 1 | 10 | 2 | 30 |
| 1 | 10 | 2 | 30 |
| 1 | 10 | 2 | 30 |
| 1 | 10 | 2 | 30 |
| 1 | 9 | 2 | 29 |
| 1 | 9 | 2 | 28 |
| 1 | 9 | 2 | 28 |
| 1 | 9 | 2 | 26 |
| 1 | 9 | 2 | 26 |
| 1 | 9 | 2 | 25 |
| 1 | 8 | 2 | 24 |
| 1 | 8 | 2 | 23 |
| 1 | 8 | 2 | 23 |
| 1 | 8 | 2 | 21 |
| 1 | 8 | 2 | 21 |
| 1 | 7 | 2 | 20 |
| 1 | 7 | 2 | 20 |
| 1 | 7 | 2 | 20 |
| 1 | 7 | 2 | 19 |
| 1 | 7 | 2 | 18 |
| 1 | 7 | 2 | 17 |
| 1 | 7 | 2 | 17 |
| 1 | 7 | 2 | 16 |
| 1 | 7 | 2 | 15 |
| 1 | 7 | 2 | 14 |
| 1 | 6 | 2 | 14 |
| 1 | 6 | 2 | 13 |
| 1 | 6 | 2 | 12 |
| 1 | 6 | 2 | 11 |
| 1 | 6 | 2 | 11 |
| 1 | 6 | 2 | 10 |
| 1 | 6 | 2 | 10 |
| 1 | 6 | 2 | 10 |
| 1 | 5 | 2 | 9 |
| 1 | 5 | 2 | 9 |
| 1 | 5 | 2 | 9 |
| 1 | 5 | 2 | 7 |
| 1 | 5 | 2 | 7 |
| 1 | 5 | 2 | 6 |
| 1 | 5 | 2 | 6 |
| 1 | 5 | 2 | 4 |
| 1 | 5 | 2 | 4 |
| 1 | 4 | 2 | 4 |
| 1 | 4 | 2 | 3 |
| 1 | 4 | 2 | 3 |
| 1 | 4 | 2 | 2 |
| 1 | 4 | 2 | 1 |
| 1 | 4 | 3 | 60 |
| 1 | 4 | 3 | 39 |
| 1 | 4 | 3 | 33 |
| 1 | 4 | 3 | 30 |
| 1 | 4 | 3 | 30 |
| 1 | 4 | 3 | 28 |
| 1 | 3 | 3 | 27 |
| 1 | 3 | 3 | 25 |
| 1 | 3 | 3 | 21 |
| 1 | 3 | 3 | 20 |
| 1 | 3 | 3 | 20 |
| 1 | 3 | 3 | 20 |
| 1 | 3 | 3 | 18 |
| 1 | 3 | 3 | 17 |
| 1 | 3 | 3 | 17 |
| 1 | 3 | 3 | 16 |
| 1 | 3 | 3 | 16 |
| 1 | 3 | 3 | 15 |
| 1 | 2 | 3 | 15 |
| 1 | 2 | 3 | 14 |
| 1 | 2 | 3 | 14 |
| 1 | 2 | 3 | 14 |
| 1 | 2 | 3 | 14 |
| 1 | 2 | 3 | 13 |
| 1 | 2 | 3 | 10 |
| 1 | 2 | 3 | 6 |
| 1 | 2 | 3 | 4 |
| 1 | 2 | 3 | 3 |
| 1 | 1 | 4 | 86 |
| 1 | 1 | 4 | 74 |
| 1 | 1 | 4 | 68 |
| 1 | 1 | 4 | 63 |
| 1 | 1 | 4 | 44 |
| 1 | 1 | 4 | 44 |
| 1 | 1 | 4 | 42 |
| 1 | 1 | 4 | 40 |
| 1 | 1 | 4 | 40 |
| 1 | 1 | 4 | 35 |
| 1 | 1 | 4 | 35 |
| 2 | 54 | 4 | 30 |
| 2 | 45 | 4 | 30 |
| 2 | 40 | 4 | 30 |
| 2 | 39 | 4 | 28 |
| 2 | 38 | 4 | 22 |
| 2 | 37 | 4 | 22 |
| 2 | 35 | 4 | 21 |
| 2 | 33 | 4 | 18 |
| 2 | 30 | 4 | 17 |
| 2 | 30 | 4 | 17 |
| 2 | 30 | 4 | 17 |
| 2 | 30 | 4 | 16 |
| 2 | 30 | 4 | 15 |
| 2 | 30 | 4 | 13 |
| 2 | 30 | 4 | 11 |
| 2 | 30 | 4 | 11 |
| 2 | 29 | 4 | 10 |
| 2 | 29 | 4 | 9 |
| 2 | 28 | 4 | 2 |
| 2 | 28 | 4 | 2 |
| 2 | 26 | 5 | 86 |
| 2 | 25 | 5 | 86 |
| 2 | 24 | 5 | 79 |
| 2 | 23 | 5 | 60 |
| 2 | 23 | 5 | 57 |
| 2 | 23 | 5 | 51 |
| 2 | 22 | 5 | 50 |
| 2 | 21 | 5 | 50 |
| 2 | 21 | 5 | 45 |
| 2 | 20 | 5 | 45 |
| 2 | 20 | 5 | 40 |
| 2 | 19 | 5 | 38 |
| 2 | 19 | 5 | 37 |
| 2 | 19 | 5 | 36 |
| 2 | 18 | 5 | 35 |
| 2 | 18 | 5 | 30 |
| 2 | 18 | 5 | 30 |
| 2 | 17 | 5 | 30 |
| 2 | 16 | 5 | 30 |
| 2 | 16 | 5 | 28 |
| 2 | 16 | 5 | 28 |
| 2 | 15 | 5 | 25 |
| 2 | 15 | 5 | 24 |
| 2 | 15 | 5 | 22 |
| 2 | 14 | 5 | 21 |
| 2 | 14 | 5 | 21 |
| 2 | 14 | 5 | 16 |
| 2 | 14 | 5 | 13 |
| 2 | 14 | 5 | 12 |
| 2 | 14 | 5 | 12 |
| 2 | 14 | 5 | 12 |
| 2 | 14 | 5 | 10 |
| 2 | 13 | 5 | 7 |
| 2 | 13 | 5 | 7 |
| 2 | 13 | 5 | 6 |
| 2 | 13 | | |
| 2 | 12 | | |
| 2 | 12 | | |
| 2 | 12 | | |
| 2 | 11 | | |
| 2 | 11 | | |
| 2 | 11 | | |
| 2 | 11 | | |
| 2 | 11 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 10 | | |
| 2 | 9 | | |
| 2 | 9 | | |
| 2 | 9 | | |
| 2 | 8 | | |
| 2 | 8 | | |
| 2 | 7 | | |
| 2 | 7 | | |
| 2 | 7 | | |
| 2 | 7 | | |
| 2 | 7 | | |
| 2 | 7 | | |
| 2 | 7 | | |
| 2 | 7 | | |
| 2 | 7 | | |
| 2 | 7 | | |
| 2 | 6 | | |
| 2 | 6 | | |
| 2 | 5 | | |
| 2 | 5 | | |
| 2 | 5 | | |
| 2 | 5 | | |
| 2 | 5 | | |
| 2 | 5 | | |
| 2 | 5 | | |
| 2 | 5 | | |
| 2 | 5 | | |
| 2 | 5 | | |
| 2 | 5 | | |
| 2 | 5 | | |
| 2 | 4 | | |
| 2 | 4 | | |
| 2 | 4 | | |
| 2 | 4 | | |
| 2 | 4 | | |
| 2 | 3 | | |
| 2 | 3 | | |
| 2 | 3 | | |
| 2 | 3 | | |
| 2 | 3 | | |
| 2 | 3 | | |
| 2 | 3 | | |
| 2 | 3 | | |
| 2 | 2 | | |
| 2 | 2 | | |
| 2 | 2 | | |
| 2 | 2 | | |
| 2 | 2 | | |
| 2 | 2 | | |
| 2 | 2 | | |
| 2 | 1 | | |
| 2 | 1 | | |
| 2 | 1 | | |
| 3 | 70 | | |
| 3 | 70 | | |
| 3 | 65 | | |
| 3 | 56 | | |
| 3 | 54 | | |
| 3 | 45 | | |
| 3 | 34 | | |
| 3 | 32 | | |
| 3 | 32 | | |
| 3 | 31 | | |
| 3 | 30 | | |
| 3 | 30 | | |
| 3 | 30 | | |
| 3 | 30 | | |
| 3 | 30 | | |
| 3 | 30 | | |
| 3 | 26 | | |
| 3 | 25 | | |
| 3 | 25 | | |
| 3 | 24 | | |
| 3 | 24 | | |
| 3 | 23 | | |
| 3 | 21 | | |
| 3 | 21 | | |
| 3 | 20 | | |
| 3 | 20 | | |
| 3 | 20 | | |
| 3 | 20 | | |
| 3 | 20 | | |
| 3 | 20 | | |
| 3 | 20 | | |
| 3 | 19 | | |
| 3 | 19 | | |
| 3 | 18 | | |
| 3 | 18 | | |
| 3 | 18 | | |
| 3 | 17 | | |
| 3 | 16 | | |
| 3 | 16 | | |
| 3 | 16 | | |
| 3 | 16 | | |
| 3 | 16 | | |
| 3 | 16 | | |
| 3 | 15 | | |
| 3 | 15 | | |
| 3 | 14 | | |
| 3 | 14 | | |
| 3 | 14 | | |
| 3 | 14 | | |
| 3 | 13 | | |
| 3 | 13 | | |
| 3 | 13 | | |
| 3 | 12 | | |
| 3 | 11 | | |
| 3 | 10 | | |
| 3 | 10 | | |
| 3 | 10 | | |
| 3 | 10 | | |
| 3 | 10 | | |
| 3 | 10 | | |
| 3 | 9 | | |
| 3 | 9 | | |
| 3 | 9 | | |
| 3 | 8 | | |
| 3 | 8 | | |
| 3 | 8 | | |
| 3 | 8 | | |
| 3 | 7 | | |
| 3 | 7 | | |
| 3 | 7 | | |
| 3 | 7 | | |
| 3 | 7 | | |
| 3 | 7 | | |
| 3 | 7 | | |
| 3 | 6 | | |
| 3 | 5 | | |
| 3 | 5 | | |
| 3 | 5 | | |
| 3 | 5 | | |
| 3 | 5 | | |
| 3 | 5 | | |
| 3 | 5 | | |
| 3 | 4 | | |
| 3 | 4 | | |
| 3 | 4 | | |
| 3 | 4 | | |
| 3 | 3 | | |
| 3 | 3 | | |
| 3 | 3 | | |
| 3 | 3 | | |
| 3 | 3 | | |
| 3 | 3 | | |
| 3 | 3 | | |
| 3 | 3 | | |
| 3 | 3 | | |
| 3 | 2 | | |
| 3 | 2 | | |
| 3 | 2 | | |
| 3 | 2 | | |
| 3 | 2 | | |
| 3 | 1 | | |
| 3 | 1 | | |
| 3 | 1 | | |
| 3 | 1 | | |
| 3 | 1 | | |
| 3 | 1 | | |
| 3 | 1 | | |
| 3 | 1 | | |
| 3 | 1 | | |
| 3 | 1 | | |
| 3 | 1 | | |
| 3 | 1 | | |
| 4 | 55 | | |
| 4 | 53 | | |
| 4 | 52 | | |
| 4 | 50 | | |
| 4 | 45 | | |
| 4 | 43 | | |
| 4 | 40 | | |
| 4 | 35 | | |
| 4 | 35 | | |
| 4 | 29 | | |
| 4 | 28 | | |
| 4 | 25 | | |
| 4 | 25 | | |
| 4 | 25 | | |
| 4 | 22 | | |
| 4 | 21 | | |
| 4 | 21 | | |
| 4 | 20 | | |
| 4 | 20 | | |
| 4 | 20 | | |
| 4 | 20 | | |
| 4 | 20 | | |
| 4 | 19 | | |
| 4 | 19 | | |
| 4 | 18 | | |
| 4 | 17 | | |
| 4 | 16 | | |
| 4 | 15 | | |
| 4 | 15 | | |
| 4 | 15 | | |
| 4 | 15 | | |
| 4 | 14 | | |
| 4 | 14 | | |
| 4 | 14 | | |
| 4 | 14 | | |
| 4 | 14 | | |
| 4 | 12 | | |
| 4 | 12 | | |
| 4 | 12 | | |
| 4 | 10 | | |
| 4 | 10 | | |
| 4 | 10 | | |
| 4 | 10 | | |
| 4 | 10 | | |
| 4 | 9 | | |
| 4 | 8 | | |
| 4 | 8 | | |
| 4 | 7 | | |
| 4 | 7 | | |
| 4 | 7 | | |
| 4 | 7 | | |
| 4 | 7 | | |
| 4 | 7 | | |
| 4 | 7 | | |
| 4 | 7 | | |
| 4 | 6 | | |
| 4 | 6 | | |
| 4 | 5 | | |
| 4 | 5 | | |
| 4 | 5 | | |
| 4 | 5 | | |
| 4 | 5 | | |
| 4 | 5 | | |
| 4 | 4 | | |
| 4 | 4 | | |
| 4 | 4 | | |
| 4 | 3 | | |
| 4 | 3 | | |
| 4 | 3 | | |
| 4 | 3 | | |
| 4 | 3 | | |
| 4 | 3 | | |
| 4 | 3 | | |
| 4 | 2 | | |
| 4 | 2 | | |
| 4 | 2 | | |
| 4 | 2 | | |
| 4 | 2 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 4 | 1 | | |
| 5 | 62 | | |
| 5 | 60 | | |
| 5 | 60 | | |
| 5 | 60 | | |
| 5 | 55 | | |
| 5 | 54 | | |
| 5 | 54 | | |
| 5 | 50 | | |
| 5 | 50 | | |
| 5 | 50 | | |
| 5 | 50 | | |
| 5 | 50 | | |
| 5 | 50 | | |
| 5 | 49 | | |
| 5 | 45 | | |
| 5 | 45 | | |
| 5 | 45 | | |
| 5 | 45 | | |
| 5 | 45 | | |
| 5 | 45 | | |
| 5 | 45 | | |
| 5 | 45 | | |
| 5 | 43 | | |
| 5 | 42 | | |
| 5 | 42 | | |
| 5 | 41 | | |
| 5 | 41 | | |
| 5 | 40 | | |
| 5 | 40 | | |
| 5 | 40 | | |
| 5 | 40 | | |
| 5 | 39 | | |
| 5 | 36 | | |
| 5 | 35 | | |
| 5 | 35 | | |
| 5 | 35 | | |
| 5 | 35 | | |
| 5 | 35 | | |
| 5 | 35 | | |
| 5 | 34 | | |
| 5 | 34 | | |
| 5 | 33 | | |
| 5 | 32 | | |
| 5 | 32 | | |
| 5 | 31 | | |
| 5 | 31 | | |
| 5 | 31 | | |
| 5 | 31 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 30 | | |
| 5 | 29 | | |
| 5 | 29 | | |
| 5 | 28 | | |
| 5 | 28 | | |
| 5 | 28 | | |
| 5 | 28 | | |
| 5 | 28 | | |
| 5 | 27 | | |
| 5 | 27 | | |
| 5 | 27 | | |
| 5 | 26 | | |
| 5 | 26 | | |
| 5 | 26 | | |
| 5 | 25 | | |
| 5 | 25 | | |
| 5 | 25 | | |
| 5 | 25 | | |
| 5 | 25 | | |
| 5 | 24 | | |
| 5 | 24 | | |
| 5 | 23 | | |
| 5 | 23 | | |
| 5 | 23 | | |
| 5 | 23 | | |
| 5 | 22 | | |
| 5 | 22 | | |
| 5 | 22 | | |
| 5 | 21 | | |
| 5 | 21 | | |
| 5 | 21 | | |
| 5 | 21 | | |
| 5 | 21 | | |
| 5 | 21 | | |
| 5 | 21 | | |
| 5 | 21 | | |
| 5 | 21 | | |
| 5 | 21 | | |
| 5 | 20 | | |
| 5 | 20 | | |
| 5 | 20 | | |
| 5 | 20 | | |
| 5 | 20 | | |
| 5 | 20 | | |
| 5 | 20 | | |
| 5 | 20 | | |
| 5 | 19 | | |
| 5 | 18 | | |
| 5 | 17 | | |
| 5 | 16 | | |
| 5 | 16 | | |
| 5 | 16 | | |
| 5 | 16 | | |
| 5 | 15 | | |
| 5 | 15 | | |
| 5 | 15 | | |
| 5 | 15 | | |
| 5 | 15 | | |
| 5 | 15 | | |
| 5 | 15 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 14 | | |
| 5 | 13 | | |
| 5 | 13 | | |
| 5 | 13 | | |
| 5 | 12 | | |
| 5 | 11 | | |
| 5 | 11 | | |
| 5 | 10 | | |
| 5 | 10 | | |
| 5 | 10 | | |
| 5 | 10 | | |
| 5 | 10 | | |
| 5 | 10 | | |
| 5 | 10 | | |
| 5 | 10 | | |
| 5 | 10 | | |
| 5 | 10 | | |
| 5 | 10 | | |
| 5 | 10 | | |
| 5 | 8 | | |
| 5 | 8 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 7 | | |
| 5 | 6 | | |
| 5 | 5 | | |
| 5 | 5 | | |
| 5 | 5 | | |
| 5 | 5 | | |
| 5 | 5 | | |
| 5 | 5 | | |
| 5 | 5 | | |
| 5 | 5 | | |
| 5 | 5 | | |
| 5 | 4 | | |
| 5 | 4 | | |
| 5 | 4 | | |
| 5 | 4 | | |
| 5 | 4 | | |
| 5 | 3 | | |
| 5 | 3 | | |
| 5 | 3 | | |
| 5 | 3 | | |
| 5 | 2 | | |
| 5 | 2 | | |
| 5 | 2 | | |
| 5 | 2 | | |
| 5 | 2 | | |
| 5 | 2 | | |
| 5 | 2 | | |
| 5 | 2 | | |
| 5 | 2 | | |
| 5 | 2 | | |
| 5 | 2 | | |
| 5 | 2 | | |
| 5 | 2 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |
| 5 | 1 | | |

| | | 1 | 2 | 3 | 4 | 5 |
|---|---|---|---|---|---|---|
| | | Frequency (Negative) | | | | |
| Percentile | 90% | 36.6 | 33.2 | 30.9 | 63 | 58.8 |
| | 75% | 25 | 28 | 25.5 | 40 | 45 |
| | 50% | 15 | 18 | 17 | 22 | 30 |
| | 25% | 7 | 9.5 | 14 | 15.5 | 18.5 |
| | 10% | 3 | 4 | 8.8 | 10 | 10.8 |
| | | Frequency (Positive) | | | | |
| Percentile | 90% | 30 | 30 | 30 | 28.9 | 40.6 |
| | 75% | 18.5 | 18.25 | 20 | 19 | 30 |
| | 50% | 10 | 10 | 10 | 8 | 14 |
| | 25% | 5 | 5 | 4 | 3 | 3 |
| | 10% | 2 | 3 | 1.1 | 1 | 1 |
| | | Frequency (Merged) | | | | |
| Percentile | 90% | 33.3 | 31.6 | 30.45 | 45.95 | 49.7 |
| | 75% | 21.75 | 23.125 | 22.75 | 29.5 | 37.5 |
| | 50% | 12.5 | 14 | 13.5 | 15 | 22 |
| | 25% | 6 | 7.25 | 9 | 9.25 | 10.75 |
| | 10% | 2.5 | 3.5 | 4.95 | 5.5 | 5.9 |

| | | Slope | Intercept |
|---|---|---|---|
| | | (m) | (b) |
| [symbol] | 90% | 4.715 | 24.055 |
| | 75% | 3.7875 | 15.5625 |
| | 50% | 2 | 9.4 |
| | 25% | 1.15 | 5 |
| | 10% | 0.88 | 1.83 |

| y = | | | |
|---|---|---|---|
| mx + b | Days | Frequency (x) | |
| 90% | 38.2 | 3 | <− Number based |
| | | | on User Selection |
| 75% | 26.925 | | (Editable) |
| 50% | 15.4 | | |
| 25% | 8.45 | | |
| 10% | 4.47 | | |

[symbol]

indicates data missing or illegible when filed

The following table describes an implementation of estimated metabolic speed in the representations of characteristics of the person:

TABLE 2
Metabolic Speed

| Test Positive | | Test Negative | | | | | | | | |
|---|---|---|---|---|---|---|---|---|---|---|
| (Fail) | | (Pass) | | | | | | | | |
| Metabolism | Days | Metabolism | Days | | | 1 | 2 | 3 | 4 | 5 |
| 1 | 54 | 2 | 86 | | | Metabolism (Negative) | | | | |
| 1 | 50 | 2 | 57 | Percentile | 90% | 0 | 49.1 | 42.2 | 40 | 30.8 |
| 1 | 45 | 2 | 50 | | 75% | 0 | 38.75 | 30 | 28 | 22.5 |
| 1 | 40 | 2 | 41 | | 50% | 0 | 29.5 | 20.5 | 17 | 16 |
| 1 | 39 | 2 | 40 | | 25% | 0 | 15.75 | 11.75 | 11 | 9 |
| 1 | 36 | 2 | 39 | | 10% | 0 | 7.1 | 5.9 | 6 | 3 |
| 1 | 35 | 2 | 38 | | | | | | | |
| 1 | 35 | 2 | 37 | | | Metabolism (Positive) | | | | |
| 1 | 20 | 2 | 36 | Percentile | 90% | 45 | 40.3 | 33.3 | 30 | 28 |
| 1 | 18 | 2 | 30 | | 75% | 36 | 22.75 | 23 | 21 | 18.5 |
| 1 | 11 | 2 | 30 | | 50% | 11 | 14 | 12 | 10 | 9 |
| 1 | 11 | 2 | 29 | | 25% | 1 | 4.25 | 5 | 4 | 3 |
| 1 | 10 | 2 | 28 | | 10% | 1 | 1 | 1 | 2 | 1 |
| 1 | 10 | 2 | 26 | | | | | | | |
| 1 | 4 | 2 | 25 | | | | | | | |
| 1 | 1 | 2 | 21 | | | Metabolism (Merged) | | | | |
| 1 | 1 | 2 | 14 | Percentile | 90% | 45 | 44.7 | 37.75 | 35 | 29.4 |
| 1 | 1 | 2 | 10 | | 75% | 36 | 30.75 | 26.5 | 24.5 | 20.5 |
| 1 | 1 | 2 | 8 | | 50% | NULL | 21.75 | 16.25 | 13.5 | 12.5 |
| 1 | 1 | 2 | 7 | | 25% | NULL | 10 | 8.375 | 7.5 | 6 |
| 1 | 1 | 2 | 7 | | 10% | NULL | 4.05 | 3.45 | 4 | 2 |
| 2 | 60 | 2 | 6 | | | | | | | |

| | | | | | |
|---|---|---|---|---|---|
| 2 | 52 | 3 | 74 | Slope | |
| | | | | (m) | Intercept (b) |

| | | | | | | | |
|---|---|---|---|---|---|---|---|
| 2 | 50 | 3 | 63 | Equations | 90% | −4.09 | 50.64 |
| 2 | 49 | 3 | 60 | | 75% | −3.725 | 38.825 |
| 2 | 45 | 3 | 60 | | 50% | −3.05 | 26.675 |
| 2 | 45 | 3 | 60 | | 25% | −1.2875 | 12.475 |
| 2 | 45 | 3 | 51 | | 10% | −0.56 | 5.335 |
| 2 | 43 | 3 | 45 | | | | |
| 2 | 42 | 3 | 45 | | | | |

| | | | | | | |
|---|---|---|---|---|---|---|
| | | | | y = | | Metabolism |
| 2 | 41 | 3 | 45 | mx + b | Days | (x) |

| | | | | | | |
|---|---|---|---|---|---|---|
| 2 | 40 | 3 | 44 | 90% | 30.19 | 5 |
| 2 | 37 | 3 | 42 | 75% | 20.2 | |
| 2 | 35 | 3 | 42 | 50% | 11.425 | |
| 2 | 35 | 3 | 40 | 25% | 6.0375 | |
| 2 | 32 | 3 | 40 | 10% | 2.535 | |
| 2 | 31 | 3 | 40 | | | |
| 2 | 30 | 3 | 38 | | | |
| 2 | 26 | 3 | 38 | | | |
| 2 | 26 | 3 | 36 | | | |
| 2 | 26 | 3 | 35 | | | |
| 2 | 25 | 3 | 35 | | | |
| 2 | 25 | 3 | 35 | | | |
| 2 | 24 | 3 | 35 | | | |
| 2 | 24 | 3 | 30 | | | |
| 2 | 23 | 3 | 30 | | | |
| 2 | 22 | 3 | 30 | | | |
| 2 | 21 | 3 | 30 | | | |
| 2 | 21 | 3 | 30 | | | |
| 2 | 21 | 3 | 30 | | | |
| 2 | 21 | 3 | 30 | | | |
| 2 | 21 | 3 | 30 | | | |
| 2 | 20 | 3 | 30 | | | |
| 2 | 20 | 3 | 28 | | | |
| 2 | 20 | 3 | 28 | | | |
| 2 | 20 | 3 | 28 | | | |
| 2 | 20 | 3 | 28 | | | |
| 2 | 20 | 3 | 26 | | | |
| 2 | 19 | 3 | 26 | | | |
| 2 | 18 | 3 | 25 | | | |
| 2 | 17 | 3 | 25 | | | |
| 2 | 17 | 3 | 24 | | | |
| 2 | 17 | 3 | 24 | | | |
| 2 | 16 | 3 | 24 | | | |
| 2 | 15 | 3 | 24 | | | |
| 2 | 15 | 3 | 23 | | | |
| 2 | 15 | 3 | 23 | | | |
| 2 | 14 | 3 | 22 | | | |
| 2 | 14 | 3 | 21 | | | |
| 2 | 14 | 3 | 21 | | | |
| 2 | 14 | 3 | 21 | | | |
| 2 | 14 | 3 | 21 | | | |
| 2 | 14 | 3 | 20 | | | |
| 2 | 13 | 3 | 20 | | | |
| 2 | 13 | 3 | 20 | | | |
| 2 | 13 | 3 | 20 | | | |
| 2 | 13 | 3 | 20 | | | |
| 2 | 12 | 3 | 19 | | | |
| 2 | 10 | 3 | 19 | | | |
| 2 | 10 | 3 | 18 | | | |
| 2 | 10 | 3 | 17 | | | |
| 2 | 10 | 3 | 17 | | | |
| 2 | 10 | 3 | 16 | | | |
| 2 | 10 | 3 | 16 | | | |
| 2 | 10 | 3 | 16 | | | |
| 2 | 10 | 3 | 15 | | | |
| 2 | 7 | 3 | 15 | | | |
| 2 | 7 | 3 | 15 | | | |
| 2 | 6 | 3 | 15 | | | |
| 2 | 6 | 3 | 14 | | | |
| 2 | 5 | 3 | 14 | | | |
| 2 | 5 | 3 | 14 | | | |
| 2 | 5 | 3 | 14 | | | |
| 2 | 5 | 3 | 13 | | | |
| 2 | 4 | 3 | 13 | | | |
| 2 | 4 | 3 | 12 | | | |
| 2 | 3 | 3 | 12 | | | |
| 2 | 3 | 3 | 11 | | | |
| 2 | 3 | 3 | 11 | | | |
| 2 | 3 | 3 | 11 | | | |
| 2 | 3 | 3 | 10 | | | |
| 2 | 3 | 3 | 10 | | | |
| 2 | 3 | 3 | 10 | | | |
| 2 | 2 | 3 | 10 | | | |
| 2 | 2 | 3 | 9 | | | |
| 2 | 2 | 3 | 9 | | | |
| 2 | 2 | 3 | 9 | | | |
| 2 | 2 | 3 | 7 | | | |
| 2 | 1 | 3 | 7 | | | |
| 2 | 1 | 3 | 6 | | | |
| 2 | 1 | 3 | 6 | | | |
| 2 | 1 | 3 | 6 | | | |
| 2 | 1 | 3 | 5 | | | |
| 2 | 1 | 3 | 5 | | | |
| 2 | 1 | 3 | 4 | | | |
| 2 | 1 | 3 | 4 | | | |
| 2 | 1 | 3 | 3 | | | |
| 2 | 1 | 3 | 3 | | | |
| 2 | 1 | 3 | 3 | | | |
| 3 | 70 | 3 | 2 | | | |
| 3 | 70 | 3 | 2 | | | |
| 3 | 65 | 3 | 2 | | | |
| 3 | 60 | 4 | 86 | | | |
| 3 | 60 | 4 | 79 | | | |
| 3 | 60 | 4 | 68 | | | |
| 3 | 56 | 4 | 50 | | | |
| 3 | 55 | 4 | 49 | | | |
| 3 | 55 | 4 | 44 | | | |
| 3 | 54 | 4 | 40 | | | |
| 3 | 53 | 4 | 40 | | | |
| 3 | 50 | 4 | 39 | | | |
| 3 | 50 | 4 | 37 | | | |
| 3 | 50 | 4 | 35 | | | |
| 3 | 50 | 4 | 34 | | | |
| 3 | 50 | 4 | 34 | | | |
| 3 | 50 | 4 | 30 | | | |
| 3 | 45 | 4 | 29 | | | |
| 3 | 45 | 4 | 29 | | | |
| 3 | 45 | 4 | 28 | | | |
| 3 | 45 | 4 | 28 | | | |
| 3 | 45 | 4 | 28 | | | |
| 3 | 45 | 4 | 28 | | | |
| 3 | 42 | 4 | 24 | | | |
| 3 | 41 | 4 | 22 | | | |
| 3 | 40 | 4 | 22 | | | |
| 3 | 40 | 4 | 22 | | | |
| 3 | 40 | 4 | 21 | | | |
| 3 | 36 | 4 | 21 | | | |
| 3 | 35 | 4 | 21 | | | |
| 3 | 35 | 4 | 20 | | | |
| 3 | 35 | 4 | 20 | | | |
| 3 | 35 | 4 | 20 | | | |
| 3 | 34 | 4 | 20 | | | |
| 3 | 34 | 4 | 18 | | | |
| 3 | 33 | 4 | 17 | | | |
| 3 | 33 | 4 | 16 | | | |
| 3 | 32 | 4 | 16 | | | |
| 3 | 32 | 4 | 16 | | | |
| 3 | 32 | 4 | 15 | | | |
| 3 | 31 | 4 | 15 | | | |
| 3 | 31 | 4 | 14 | | | |
| 3 | 31 | 4 | 13 | | | |
| 3 | 31 | 4 | 13 | | | |
| 3 | 31 | 4 | 13 | | | |
| 3 | 30 | 4 | 13 | | | |
| 3 | 30 | 4 | 12 | | | |
| 3 | 30 | 4 | 12 | | | |
| 3 | 30 | 4 | 11 | | | |
| 3 | 30 | 4 | 11 | | | |
| 3 | 30 | 4 | 11 | | | |
| 3 | 30 | 4 | 11 | | | |
| 3 | 30 | 4 | 10 | | | |
| 3 | 30 | 4 | 10 | | | |
| 3 | 30 | 4 | 9 | | | |
| 3 | 30 | 4 | 9 | | | |
| 3 | 30 | 4 | 7 | | | |
| 3 | 30 | 4 | 7 | | | |
| 3 | 30 | 4 | 7 | | | |
| 3 | 30 | 4 | 6 | | | |
| 3 | 30 | 4 | 6 | | | |
| 3 | 30 | 4 | 6 | | | |
| 3 | 30 | 4 | 4 | | | |
| 3 | 30 | 4 | 3 | | | |
| 3 | 30 | 4 | 2 | | | |
| 3 | 30 | 4 | 2 | | | |
| 3 | 30 | 4 | 2 | | | |
| 3 | 30 | 4 | 1 | | | |
| 3 | 29 | 5 | 86 | | | |
| 3 | 29 | 5 | 45 | | | |
| 3 | 29 | 5 | 35 | | | |
| 3 | 28 | 5 | 33 | | | |
| 3 | 28 | 5 | 32 | | | |
| 3 | 28 | 5 | 30 | | | |
| 3 | 28 | 5 | 30 | | | |
| 3 | 28 | 5 | 30 | | | |
| 3 | 28 | 5 | 27 | | | |
| 3 | 26 | 5 | 25 | | | |
| 3 | 25 | 5 | 25 | | | |
| 3 | 25 | 5 | 23 | | | |
| 3 | 25 | 5 | 22 | | | |
| 3 | 25 | 5 | 22 | | | |
| 3 | 24 | 5 | 21 | | | |
| 3 | 24 | 5 | 20 | | | |
| 3 | 24 | 5 | 18 | | | |
| 3 | 24 | 5 | 18 | | | |
| 3 | 23 | 5 | 17 | | | |
| 3 | 23 | 5 | 17 | | | |
| 3 | 23 | 5 | 17 | | | |
| 3 | 22 | 5 | 17 | | | |
| 3 | 21 | 5 | 17 | | | |
| 3 | 21 | 5 | 16 | | | |
| 3 | 21 | 5 | 14 | | | |
| 3 | 21 | 5 | 14 | | | |
| 3 | 21 | 5 | 13 | | | |
| 3 | 20 | 5 | 12 | | | |
| 3 | 20 | 5 | 12 | | | |
| 3 | 20 | 5 | 12 | | | |
| 3 | 20 | 5 | 12 | | | |
| 3 | 20 | 5 | 10 | | | |
| 3 | 20 | 5 | 10 | | | |
| 3 | 20 | 5 | 9 | | | |
| 3 | 20 | 5 | 9 | | | |
| 3 | 20 | 5 | 9 | | | |
| 3 | 20 | 5 | 7 | | | |
| 3 | 20 | 5 | 7 | | | |
| 3 | 20 | 5 | 6 | | | |
| 3 | 20 | 5 | 4 | | | |
| 3 | 19 | 5 | 3 | | | |
| 3 | 19 | 5 | 3 | | | |
| 3 | 19 | 5 | 3 | | | |
| 3 | 19 | 5 | 3 | | | |
| 3 | 19 | 5 | 3 | | | |
| 3 | 19 | 5 | 1 | | | |
| 3 | 19 | 5 | 1 | | | |
| 3 | 18 | | | | | |
| 3 | 18 | | | | | |
| 3 | 18 | | | | | |
| 3 | 18 | | | | | |
| 3 | 18 | | | | | |
| 3 | 17 | | | | | |
| 3 | 17 | | | | | |
| 3 | 17 | | | | | |
| 3 | 16 | | | | | |
| 3 | 16 | | | | | |
| 3 | 16 | | | | | |
| 3 | 16 | | | | | |
| 3 | 16 | | | | | |
| 3 | 16 | | | | | |
| 3 | 16 | | | | | |
| 3 | 16 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 15 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 14 | | | | | |
| 3 | 13 | | | | | |
| 3 | 13 | | | | | |
| 3 | 13 | | | | | |
| 3 | 13 | | | | | |
| 3 | 13 | | | | | |
| 3 | 12 | | | | | |
| 3 | 12 | | | | | |
| 3 | 12 | | | | | |
| 3 | 12 | | | | | |
| 3 | 12 | | | | | |
| 3 | 12 | | | | | |
| 3 | 12 | | | | | |
| 3 | 12 | | | | | |
| 3 | 12 | | | | | |
| 3 | 11 | | | | | |
| 3 | 11 | | | | | |
| 3 | 11 | | | | | |
| 3 | 11 | | | | | |
| 3 | 11 | | | | | |
| 3 | 11 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 10 | | | | | |
| 3 | 9 | | | | | |
| 3 | 9 | | | | | |
| 3 | 9 | | | | | |
| 3 | 9 | | | | | |
| 3 | 9 | | | | | |
| 3 | 9 | | | | | |
| 3 | 9 | | | | | |
| 3 | 8 | | | | | |
| 3 | 8 | | | | | |
| 3 | 8 | | | | | |
| 3 | 8 | | | | | |
| 3 | 8 | | | | | |
| 3 | 8 | | | | | |
| 3 | 8 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 7 | | | | | |
| 3 | 6 | | | | | |
| 3 | 6 | | | | | |
| 3 | 6 | | | | | |
| 3 | 6 | | | | | |
| 3 | 6 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 5 | | | | | |
| 3 | 4 | | | | | |
| 3 | 4 | | | | | |
| 3 | 4 | | | | | |
| 3 | 4 | | | | | |
| 3 | 4 | | | | | |
| 3 | 4 | | | | | |
| 3 | 4 | | | | | |
| 3 | 4 | | | | | |
| 3 | 4 | | | | | |
| 3 | 4 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 3 | | | | | |
| 3 | 2 | | | | | |
| 3 | 2 | | | | | |
| 3 | 2 | | | | | |
| 3 | 2 | | | | | |
| 3 | 2 | | | | | |
| 3 | 2 | | | | | |
| 3 | 2 | | | | | |
| 3 | 2 | | | | | |
| 3 | 2 | | | | | |
| 3 | 2 | | | | | |
| 3 | 2 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 3 | 1 | | | | | |
| 4 | 62 | | | | | |
| 4 | 57 | | | | | |
| 4 | 54 | | | | | |
| 4 | 45 | | | | | |
| 4 | 45 | | | | | |
| 4 | 45 | | | | | |
| 4 | 42 | | | | | |
| 4 | 39 | | | | | |
| 4 | 38 | | | | | |
| 4 | 35 | | | | | |
| 4 | 35 | | | | | |
| 4 | 33 | | | | | |
| 4 | 32 | | | | | |
| 4 | 32 | | | | | |
| 4 | 30 | | | | | |
| 4 | 30 | | | | | |
| 4 | 30 | | | | | |
| 4 | 30 | | | | | |
| 4 | 30 | | | | | |
| 4 | 30 | | | | | |
| 4 | 30 | | | | | |
| 4 | 30 | | | | | |
| 4 | 30 | | | | | |
| 4 | 30 | | | | | |
| 4 | 30 | | | | | |
| 4 | 30 | | | | | |
| 4 | 29 | | | | | |
| 4 | 28 | | | | | |
| 4 | 26 | | | | | |
| 4 | 26 | | | | | |
| 4 | 25 | | | | | |
| 4 | 25 | | | | | |
| 4 | 25 | | | | | |
| 4 | 25 | | | | | |
| 4 | 25 | | | | | |
| 4 | 24 | | | | | |
| 4 | 24 | | | | | |
| 4 | 24 | | | | | |
| 4 | 24 | | | | | |
| 4 | 23 | | | | | |
| 4 | 22 | | | | | |
| 4 | 22 | | | | | |
| 4 | 22 | | | | | |
| 4 | 22 | | | | | |
| 4 | 21 | | | | | |
| 4 | 21 | | | | | |
| 4 | 21 | | | | | |
| 4 | 20 | | | | | |
| 4 | 20 | | | | | |
| 4 | 20 | | | | | |
| 4 | 20 | | | | | |
| 4 | 20 | | | | | |
| 4 | 19 | | | | | |
| 4 | 19 | | | | | |
| 4 | 19 | | | | | |
| 4 | 18 | | | | | |
| 4 | 16 | | | | | |
| 4 | 16 | | | | | |
| 4 | 16 | | | | | |
| 4 | 16 | | | | | |
| 4 | 16 | | | | | |
| 4 | 16 | | | | | |
| 4 | 15 | | | | | |
| 4 | 15 | | | | | |
| 4 | 15 | | | | | |
| 4 | 15 | | | | | |
| 4 | 15 | | | | | |
| 4 | 14 | | | | | |
| 4 | 14 | | | | | |
| 4 | 14 | | | | | |
| 4 | 14 | | | | | |
| 4 | 14 | | | | | |
| 4 | 14 | | | | | |
| 4 | 14 | | | | | |
| 4 | 14 | | | | | |
| 4 | 14 | | | | | |
| 4 | 14 | | | | | |
| 4 | 13 | | | | | |
| 4 | 13 | | | | | |
| 4 | 13 | | | | | |
| 4 | 13 | | | | | |
| 4 | 12 | | | | | |
| 4 | 11 | | | | | |
| 4 | 11 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 10 | | | | | |
| 4 | 9 | | | | | |
| 4 | 9 | | | | | |
| 4 | 9 | | | | | |
| 4 | 8 | | | | | |
| 4 | 8 | | | | | |
| 4 | 8 | | | | | |
| 4 | 8 | | | | | |
| 4 | 8 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 7 | | | | | |
| 4 | 6 | | | | | |
| 4 | 6 | | | | | |
| 4 | 6 | | | | | |
| 4 | 6 | | | | | |
| 4 | 5 | | | | | |
| 4 | 5 | | | | | |
| 4 | 5 | | | | | |
| 4 | 5 | | | | | |
| 4 | 5 | | | | | |
| 4 | 5 | | | | | |
| 4 | 5 | | | | | |
| 4 | 5 | | | | | |
| 4 | 4 | | | | | |
| 4 | 4 | | | | | |
| 4 | 4 | | | | | |
| 4 | 4 | | | | | |
| 4 | 4 | | | | | |
| 4 | 4 | | | | | |
| 4 | 4 | | | | | |
| 4 | 4 | | | | | |
| 4 | 4 | | | | | |
| 4 | 4 | | | | | |
| 4 | 3 | | | | | |
| 4 | 3 | | | | | |
| 4 | 3 | | | | | |
| 4 | 3 | | | | | |
| 4 | 3 | | | | | |
| 4 | 3 | | | | | |
| 4 | 3 | | | | | |
| 4 | 3 | | | | | |
| 4 | 3 | | | | | |
| 4 | 3 | | | | | |
| 4 | 2 | | | | | |
| 4 | 2 | | | | | |
| 4 | 2 | | | | | |
| 4 | 2 | | | | | |
| 4 | 2 | | | | | |
| 4 | 2 | | | | | |
| 4 | 2 | | | | | |
| 4 | 2 | | | | | |
| 4 | 2 | | | | | |
| 4 | 2 | | | | | |
| 4 | 2 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 4 | 1 | | | | | |
| 5 | 54 | | | | | |
| 5 | 45 | | | | | |
| 5 | 43 | | | | | |
| 5 | 40 | | | | | |
| 5 | 34 | | | | | |
| 5 | 33 | | | | | |
| 5 | 30 | | | | | |
| 5 | 30 | | | | | |
| 5 | 30 | | | | | |
| 5 | 30 | | | | | |
| 5 | 30 | | | | | |
| 5 | 30 | | | | | |
| 5 | 29 | | | | | |
| 5 | 28 | | | | | |
| 5 | 28 | | | | | |
| 5 | 27 | | | | | |
| 5 | 27 | | | | | |
| 5 | 27 | | | | | |
| 5 | 25 | | | | | |
| 5 | 25 | | | | | |
| 5 | 23 | | | | | |
| 5 | 23 | | | | | |
| 5 | 23 | | | | | |
| 5 | 23 | | | | | |
| 5 | 23 | | | | | |
| 5 | 21 | | | | | |
| 5 | 21 | | | | | |
| 5 | 21 | | | | | |
| 5 | 21 | | | | | |
| 5 | 21 | | | | | |
| 5 | 20 | | | | | |
| 5 | 20 | | | | | |
| 5 | 20 | | | | | |
| 5 | 20 | | | | | |
| 5 | 19 | | | | | |
| 5 | 18 | | | | | |
| 5 | 18 | | | | | |
| 5 | 16 | | | | | |
| 5 | 16 | | | | | |
| 5 | 16 | | | | | |
| 5 | 16 | | | | | |
| 5 | 15 | | | | | |
| 5 | 15 | | | | | |
| 5 | 15 | | | | | |
| 5 | 15 | | | | | |
| 5 | 14 | | | | | |
| 5 | 14 | | | | | |
| 5 | 14 | | | | | |
| 5 | 14 | | | | | |
| 5 | 14 | | | | | |
| 5 | 14 | | | | | |
| 5 | 14 | | | | | |
| 5 | 14 | | | | | |
| 5 | 14 | | | | | |
| 5 | 14 | | | | | |
| 5 | 13 | | | | | |
| 5 | 12 | | | | | |
| 5 | 12 | | | | | |
| 5 | 12 | | | | | |
| 5 | 12 | | | | | |
| 5 | 10 | | | | | |
| 5 | 10 | | | | | |
| 5 | 10 | | | | | |
| 5 | 10 | | | | | |
| 5 | 10 | | | | | |
| 5 | 10 | | | | | |
| 5 | 10 | | | | | |
| 5 | 10 | | | | | |
| 5 | 10 | | | | | |
| 5 | 9 | | | | | |
| 5 | 9 | | | | | |
| 5 | 9 | | | | | |
| 5 | 8 | | | | | |
| 5 | 8 | | | | | |
| 5 | 8 | | | | | |
| 5 | 7 | | | | | |
| 5 | 7 | | | | | |
| 5 | 7 | | | | | |
| 5 | 7 | | | | | |
| 5 | 7 | | | | | |
| 5 | 7 | | | | | |
| 5 | 7 | | | | | |
| 5 | 7 | | | | | |
| 5 | 6 | | | | | |
| 5 | 6 | | | | | |
| 5 | 6 | | | | | |
| 5 | 5 | | | | | |
| 5 | 5 | | | | | |
| 5 | 5 | | | | | |
| 5 | 5 | | | | | |
| 5 | 5 | | | | | |
| 5 | 5 | | | | | |
| 5 | 5 | | | | | |
| 5 | 5 | | | | | |
| 5 | 5 | | | | | |
| 5 | 4 | | | | | |
| 5 | 4 | | | | | |
| 5 | 4 | | | | | |
| 5 | 4 | | | | | |
| 5 | 4 | | | | | |
| 5 | 3 | | | | | |
| 5 | 3 | | | | | |
| 5 | 3 | | | | | |
| 5 | 3 | | | | | |
| 5 | 3 | | | | | |
| 5 | 3 | | | | | |
| 5 | 3 | | | | | |
| 5 | 3 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 2 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |
| 5 | 1 | | | | | |

The following table represents one of many drug test passing probability calculations in the representations of both marijuana/cannabis use and characteristics of a person:

TABLE 3
Drug Test Passing Probability Calculations

| | Gender | Weight (Lbs) | Waist (Inches) |
|---|---|---|---|
| 0 = Male, 1 = Female | 0 | 165 | 30 |

| Body Fat | | | | | |
|---|---|---|---|---|---|
| Percent | Body Fat | Frequency | Metabolism | Potency | Amount |
| 7.6% | 12.6 | 1 | 4 | 4 | 2 |

| | 1-Feb-13 | | | | | |
|---|---|---|---|---|---|---|
| Confidence Interval | Days | Days | Days | Days | Days | THC Calc |
| 90% | 31.9 | 28.8 | 34.3 | 45.8 | 35.4 | 33.0 |
| 75% | 21.4 | 19.4 | 23.9 | 29.3 | 23.6 | 22.2 |
| 50% | 13.0 | 11.4 | 14.5 | 19.6 | 14.7 | 13.6 |
| 25% | 6.8 | 6.2 | 7.3 | 16.3 | 7.2 | 7.7 |
| 10% | 3.1 | 2.7 | 3.1 | 5.4 | 4.0 | 3.3 |

Percent Weight (based off of Adj. R² values)

| Body Fat | Frequency | Metabolism | Potency | Amount |
|---|---|---|---|---|
| 0.261183081 | 0.3414 | 0.1752771 | 0.098354074 | 0.1238 |

FIG. 3 illustrates a graph 300 of the percentage of the likelihood, according to an implementation. The graph visually depicts the likelihood for a 25 year old Male, 170 (Lbs) with 32 inch waist as follows:

| Days | Percent |
|---|---|
| 31.2 | 90 |
| 24.2 | 75 |
| 17.3 | 50 |
| 12.2 | 25 |
| 8.3 | 10 |

The graph 300 is merely one implementation of many possible graphs.

### Hardware and Operating Environment

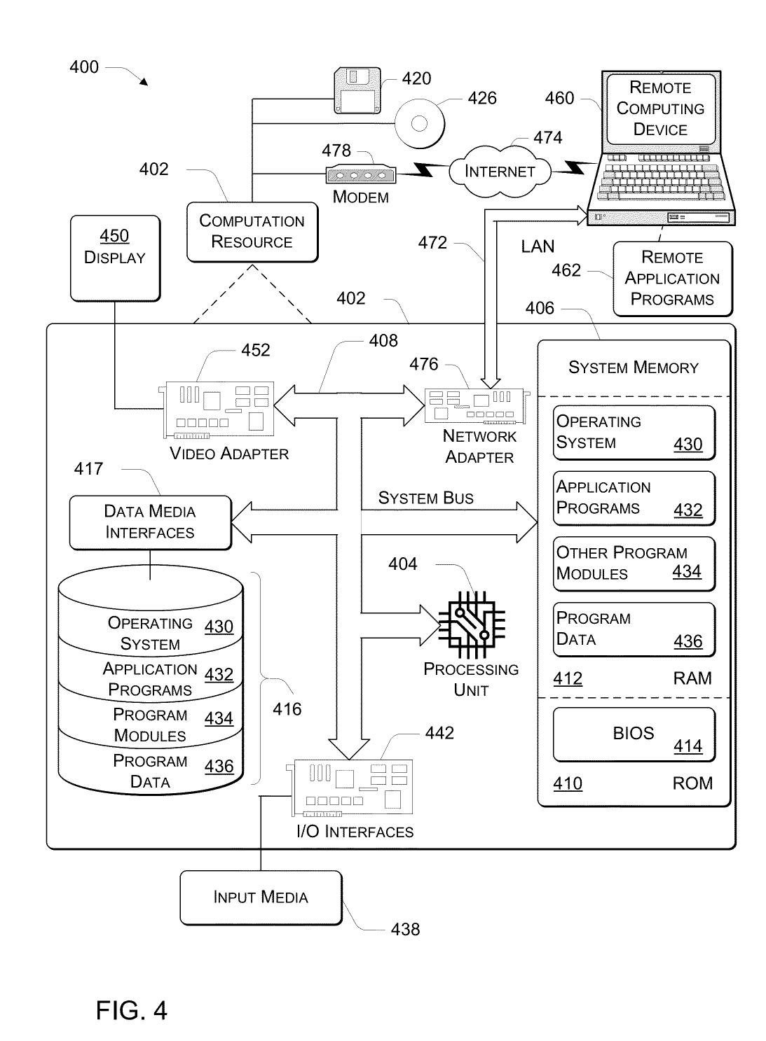

FIG. 4 is a block diagram of a hardware and operating environment 400 in which different implementations can be practiced. The description of FIG. 4 provides an overview of computer hardware and a suitable computing environment in conjunction with which some implementations can be implemented. Implementations are described in terms of a computer executing computer-executable instructions. However, some implementations can be implemented entirely in computer hardware in which the computer-executable instructions are implemented in read-only memory. Some implementations can also be implemented in client/server computing environments where remote devices that perform tasks are linked through a communications network. Program modules can be located in both local and remote memory storage devices in a distributed computing environment.

FIG. 4 illustrates an example of a general computer environment 400, in accordance with an implementation of the disclosed subject matter. The general computer environment 400 includes a computation device 402 capable of implementing the processes described herein. It will be appreciated that other devices can alternatively used that include more components, or fewer components, than those illustrated in FIG. 4.

The illustrated operating environment 400 is only one example of a suitable operating environment, and the example described with reference to FIG. 4 is not intended to suggest any limitation as to the scope of use or functionality of the implementations of this disclosure. Other well-known computing systems, environments, and/or configurations can be suitable for implementation and/or application of the subject matter disclosed herein.

The computation device 402 includes one or more processors or processing units 404, a system memory 406, and a bus 408 that couples various system components including the system memory 406 to processing unit(s) 404 and other elements in the environment 400. The bus 408 represents one or more of any of several types of bus structures, including a memory bus or memory controller, a peripheral bus, an accelerated graphics port and a processor or local bus using any of a variety of bus architectures, and can be compatible with SCSI (small computer system interconnect), or other conventional bus architectures and protocols.

The system memory 406 includes nonvolatile read-only memory (ROM) 410 and random access memory (RAM) 412, which can or can not include volatile memory elements. A basic input/output system (BIOS) 414, containing the elementary routines that help to transfer information between elements within computation device 402 and with external items, typically invoked into operating memory during start-up, is stored in ROM 410.

The computation device 402 further can include a non-volatile read/write memory 416, represented in FIG. 4 as a hard disk drive, coupled to bus 408 via a data media interface 417 (e.g., a SCSI, ATA, or other type of interface); a magnetic disk drive (not shown) for reading from, and/or writing to, a removable magnetic disk 420 and an optical disk drive (not shown) for reading from, and/or writing to, a removable optical disk 426 such as a CD, DVD, or other optical media.

The non-volatile read/write memory 416 and associated computer-readable media provide nonvolatile storage of computer-readable instructions, data structures, program modules and other data for the computation device 402. Although the exemplary environment 400 is described herein as employing a non-volatile read/write memory 416, a removable magnetic disk 420 and a removable optical disk 426, it will be appreciated by those skilled in the art that other types of computer-readable media which can store data that is accessible by a computer, such as magnetic cassettes, FLASH memory cards, random access memories (RAMs), read only memories (ROM), and the like, can also be used in the exemplary operating environment.

A number of program modules can be stored via the non-volatile read/write memory 416, magnetic disk 420, optical disk 426, ROM 410, or RAM 412, including an operating system 430, one or more application programs 432, other program modules 434 and program data 436. Examples of computer operating systems conventionally employed for some types of three-dimensional and/or two-dimensional medical image data include the NUCLEUS® operating system, the LINUX® operating system, and others, for example, providing capability for supporting application programs 432 using, for example, code modules written in the C++® computer programming language.

A user can enter commands and information into computation device 402 through input devices such as input media 438 (e.g., keyboard/keypad, tactile input or pointing device, mouse, foot-operated switching apparatus, joystick, touchscreen or touchpad, microphone, antenna etc.). Such input devices 438 are coupled to the processing unit 404 through a conventional input/output interface 442 that is, in turn, coupled to the system bus. A monitor 450 or other type of display device is also coupled to the system bus 408 via an interface, such as a video adapter 452.

The computation device 402 can include capability for operating in a networked environment using logical connections to one or more remote computers, such as a remote computer 460. The remote computer 460 can be a personal computer, a server, a router, a network PC, a peer device or other common network node, and typically includes many or all of the elements described above relative to the computation device 402. In a networked environment, program modules depicted relative to the computation device 402, or portions thereof, can be stored in a remote memory storage device such as can be associated with the remote computer 460. By way of example, remote application programs 462 reside on a memory device of the remote computer 460. The logical connections represented in FIG. 4 can include interface capabilities a storage area network (SAN, not illustrated in FIG. 4), local area network (LAN) 472 and/or a wide area network (WAN) 474, but can also include other networks.

Such networking environments are commonplace in modern computer systems, and in association with intranets and the Internet. In certain implementations, the computation device 402 executes an Internet Web browser program (which can optionally be integrated into the operating system 430), such as the “Internet Explorer®” Web browser manufactured and distributed by the Microsoft Corporation of Redmond, Wash.

When used in a LAN-coupled environment, the computation device 402 communicates with or through the local area network 472 via a network interface or adapter 476. When used in a WAN-coupled environment, the computation device 402 typically includes interfaces, such as a modem 478, or other apparatus, for establishing communications with or through the WAN 474, such as the Internet. The modem 478, which can be internal or external, is coupled to the system bus 408 via a serial port interface.

In a networked environment, program modules depicted relative to the computation device 402, or portions thereof, can be stored in remote memory apparatus. It will be appreciated that the network connections shown are exemplary, and other means of establishing a communications link between various computer systems and elements can be used.

A user of a computer can operate in a networked environment 400 using logical connections to one or more remote computers, such as a remote computer 460, which can be a personal computer, a server, a router, a network PC, a peer device or other common network node. Typically, a remote computer 460 includes many or all of the elements described above relative to the computer 400 of FIG. 4.

The computation device 402 typically includes at least some form of computer-readable media. Computer-readable media can be any available media that can be accessed by the computation device 402. By way of example, and not limitation, computer-readable media can comprise computer storage media and communication media.

Computer storage media include volatile and nonvolatile, removable and non-removable media, implemented in any method or technology for storage of information, such as computer-readable instructions, data structures, program modules or other data. The term “computer storage media” includes, but is not limited to, RAM, ROM, EEPROM, FLASH memory or other memory technology, CD, DVD, or other optical storage, magnetic cassettes, magnetic tape, magnetic disk storage or other magnetic storage devices, or any other media which can be used to store computer-intelligible information and which can be accessed by the computation device 402.

Communication media typically embodies computer-readable instructions, data structures, program modules or other data, represented via, and determinable from, a modulated data signal, such as a carrier wave or other transport mechanism, and includes any information delivery media. The term “modulated data signal” means a signal that has one or more of its characteristics set or changed in such a manner as to encode information in the signal in a fashion amenable to computer interpretation.

By way of example, and not limitation, communication media include wired media, such as wired network or direct-wired connections, and wireless media, such as acoustic, RF, infrared and other wireless media. The scope of the term computer-readable media includes combinations of any of the above.

Apparatus 200 components can be embodied as computer hardware circuitry or as a computer-readable program, or a combination of both. In another implementation, Apparatus 200 is implemented in an application service provider (ASP) system. More specifically, in the computer-readable program implementation, the programs can be structured in an object-orientation using an object-oriented language such as Java, Smalltalk or C++, and the programs can be structured in a procedural-orientation using a procedural language such as COBOL or C. The software components communicate in any of a number of means that are well-known to those skilled in the art, such as application program interfaces (API) or interprocess communication techniques such as remote procedure call (RPC), common object request broker architecture (CORBA), Component Object Model (COM), Distributed Component Object Model (DCOM), Distributed System Object Model (DSOM) and Remote Method Invocation (RMI). The components execute on as few as one computer as in general computer environment 400 in FIG. 4, or on at least as many computers as there are components.

The implementations described below generally relate to a mobile wireless communication device, hereafter referred to as a mobile device, which can be configured according to an IT policy. It should be noted that the term IT policy, in general, refers to a collection of IT policy rules, in which the IT policy rules can be defined as being either grouped or non-grouped and global or per-user. The terms grouped, non-grouped, global and per-user are defined further below. Examples of applicable communication devices include pagers, cellular phones, cellular smart-phones, wireless organizers, personal digital assistants, computers, laptops, handheld wireless communication devices, wirelessly enabled notebook computers and the like.

FIG. 5 is a block diagram of a mobile device 500, according to an implementation. The mobile device is a two-way communication device with advanced data communication capabilities including the capability to communicate with other mobile devices or computer systems through a network of transceiver stations. The mobile device may also have the capability to allow voice communication. Depending on the functionality provided by the mobile device, it may be referred to as a data messaging device, a two-way pager, a cellular telephone with data messaging capabilities, a wireless Internet appliance, or a data communication device (with or without telephony capabilities).

The mobile device 500 includes a number of components such as a main processor 502 that controls the overall operation of the mobile device 500. Communication functions, including data and voice communications, are performed through a communication subsystem 504. The communication subsystem 504 receives messages from and sends messages to wireless networks 505. Other implementations of the mobile device 500, the communication subsystem 504 can be configured in accordance with the Global System for Mobile Communication (GSM), General Packet Radio Services (GPRS), Enhanced Data GSM Environment (EDGE), Universal Mobile Telecommunications Service (UMTS), data-centric wireless networks, voice-centric wireless networks, and dual-mode networks that can support both voice and data communications over the same physical base stations. Combined dual-mode networks include, but are not limited to, Code Division Multiple Access (CDMA) or CDMA2000 networks, GSM/GPRS networks (as mentioned above), and future third-generation (3G) networks like EDGE and UMTS. Some other examples of data-centric networks include Mobitex™ and DataTAC™ network communication systems. Examples of other voice-centric data networks include Personal Communication Systems (PCS) networks like GSM and Time Division Multiple Access (TDMA) systems.

The wireless link connecting the communication subsystem 504 with the wireless network 505 represents one or more different Radio Frequency (RF) channels. With newer network protocols, these channels are capable of supporting both circuit switched voice communications and packet switched data communications.

The main processor 502 also interacts with additional subsystems such as a Random Access Memory (RAM) 506, a flash memory 508, a display 510, an auxiliary input/output (I/O) subsystem 512, a data port 514, a keyboard 516, a speaker 518, a microphone 520, short-range communications 522 and other device subsystems 524. The drug-test related data 202 is received by the communication subsystem 504 and transferred by the main processor 502 to the flash memory 508.

Some of the subsystems of the mobile device 500 perform communication-related functions, whereas other subsystems may provide “resident” or on-device functions. By way of example, the display 510 and the keyboard 516 may be used for both communication-related functions, such as entering a text message for transmission over the wireless network 505, and device-resident functions such as a calculator or task list.

The mobile device 500 can transmit and receive communication signals over the wireless network 505 after required network registration or activation procedures have been completed. Network access is associated with a subscriber or user of the mobile device 500. To identify a subscriber, the mobile device 500 requires a SIM/RUIM card 526 (i.e. Subscriber Identity Module or a Removable User Identity Module) to be inserted into a SIM/RUIM interface 528 in order to communicate with a network. The SIM card or RUIM 526 is one type of a conventional “smart card” that can be used to identify a subscriber of the mobile device 500 and to personalize the mobile device 500, among other things. Without the SIM card 526, the mobile device 500 is not fully operational for communication with the wireless network 505. By inserting the SIM card/RUIM 526 into the SIM/RUIM interface 528, a subscriber can access all subscribed services. Services may include: web browsing and messaging such as e-mail, voice mail, Short Message Service (SMS), and Multimedia Messaging Services (MMS). More advanced services may include: point of sale, field service and sales force automation. The SIM card/RUIM 526 includes a processor and memory for storing information. Once the SIM card/RUIM 526 is inserted into the SIM/RUIM interface 528, it is coupled to the main processor 502. In order to identify the subscriber, the SIM card/RUIM 526 can include some user parameters such as an International Mobile Subscriber Identity (IMSI). An advantage of using the SIM card/RUIM 526 is that a subscriber is not necessarily bound by any single physical mobile device. The SIM card/RUIM 526 may store additional subscriber information for a mobile device as well, including datebook (or calendar) information and recent call information. Alternatively, user identification information can also be programmed into the flash memory 508.

The mobile device 500 is a battery-powered device and includes a battery interface 532 for receiving one or more rechargeable batteries 530. In one or more implementations, the battery 530 can be a smart battery with an embedded microprocessor. The battery interface 532 is coupled to a regulator 533, which assists the battery 530 in providing power V+ to the mobile device 500. Although current technology makes use of a battery, future technologies such as micro fuel cells may provide the power to the mobile device 500.

The mobile device 500 also includes an operating system 534 and software components 536 to 546 which are described in more detail below. The operating system 534 and the software components 536 to 546 that are executed by the main processor 502 are typically stored in a persistent store such as the flash memory 508, which may alternatively be a read-only memory (ROM) or similar storage element (not shown). Those skilled in the art will appreciate that portions of the operating system 534 and the software components 536 to 546, such as specific device applications, or parts thereof, may be temporarily loaded into a volatile store such as the RAM 506. Other software components can also be included.

The subset of software applications 536 that control basic device operations, including data and voice communication applications, will normally be installed on the mobile device 500 during its manufacture. Other software applications include a message application 538 that can be any suitable software program that allows a user of the mobile device 500 to transmit and receive electronic messages. Various alternatives exist for the message application 538 as is well known to those skilled in the art. Messages that have been sent or received by the user are typically stored in the flash memory 508 of the mobile device 500 or some other suitable storage element in the mobile device 500. In one or more implementations, some of the sent and received messages may be stored remotely from the device 500 such as in a data store of an associated host system with which the mobile device 500 communicates.

The software applications can further include a device state module 540, a Personal Information Manager (PIM) 542, and other suitable modules (not shown). The device state module 540 provides persistence, i.e. the device state module 540 ensures that important device data is stored in persistent memory, such as the flash memory 508, so that the data is not lost when the mobile device 500 is turned off or loses power.

The PIM 542 includes functionality for organizing and managing data items of interest to the user, such as, but not limited to, e-mail, contacts, calendar events, voice mails, appointments, and task items. A PIM application has the ability to transmit and receive data items via the wireless network 505. PIM data items may be seamlessly integrated, synchronized, and updated via the wireless network 505 with the mobile device subscriber's corresponding data items stored and/or associated with a host computer system. This functionality creates a mirrored host computer on the mobile device 500 with respect to such items. This can be particularly advantageous when the host computer system is the mobile device subscriber's office computer system.

The mobile device 500 also includes a connect module 544, and an IT policy module 546. The connect module 544 implements the communication protocols that are required for the mobile device 500 to communicate with the wireless infrastructure and any host system, such as an enterprise system, with which the mobile device 500 is authorized to interface. Examples of a wireless infrastructure and an enterprise system are given in FIGS. 21 and 22, which are described in more detail below.

The connect module 544 includes a set of APIs that can be integrated with the mobile device 500 to allow the mobile device 500 to use any number of services associated with the enterprise system. The connect module 544 allows the mobile device 500 to establish an end-to-end secure, authenticated communication pipe with the host system. A subset of applications for which access is provided by the connect module 544 can be used to pass IT policy commands from the host system to the mobile device 500. This can be done in a wireless or wired manner. These instructions can then be passed to the IT policy module 546 to modify the configuration of the device 500. Alternatively, in some cases, the IT policy update can also be done over a wired connection.

The IT policy module 546 receives IT policy data that encodes the IT policy. The IT policy module 546 then ensures that the IT policy data is authenticated by the mobile device 500. The IT policy data can then be stored in the flash memory 506 in its native form. After the IT policy data is stored, a global notification can be sent by the IT policy module 546 to all of the applications residing on the mobile device 500. Applications for which the IT policy may be applicable then respond by reading the IT policy data to look for IT policy rules that are applicable.

The IT policy module 546 can include a parser 547, which can be used by the applications to read the IT policy rules. In some cases, another module or application can provide the parser. Grouped IT policy rules, described in more detail below, are retrieved as byte streams, which are then sent (recursively) into the parser to determine the values of each IT policy rule defined within the grouped IT policy rule. In one or more implementations, the IT policy module 546 can determine which applications are affected by the IT policy data and transmit a notification to only those applications. In either of these cases, for applications that are not being executed by the main processor 502 at the time of the notification, the applications can call the parser or the IT policy module 546 when the applications are executed to determine if there are any relevant IT policy rules in the newly received IT policy data.

After the IT policy rules have been applied to the applicable applications or configuration files, the IT policy module 546 sends an acknowledgement back to the host system to indicate that the IT policy data was received and successfully applied.

Other types of software applications can also be installed on the mobile device 500. These software applications can be third party applications, which are added after the manufacture of the mobile device 500. Examples of third party applications include games, calculators, utilities, etc.

The additional applications can be loaded onto the mobile device 500 through at least one of the wireless network 505, the auxiliary I/O subsystem 512, the data port 514, the short-range communications subsystem 522, or any other suitable device subsystem 524. This flexibility in application installation increases the functionality of the mobile device 500 and may provide enhanced on-device functions, communication-related functions, or both. For example, secure communication applications may enable electronic commerce functions and other such financial transactions to be performed using the mobile device 500.

The data port 514 enables a subscriber to set preferences through an external device or software application and extends the capabilities of the mobile device 500 by providing for information or software downloads to the mobile device 500 other than through a wireless communication network. The alternate download path may, for example, be used to load an encryption key onto the mobile device 500 through a direct and thus reliable and trusted connection to provide secure device communication.

The data port 514 can be any suitable port that enables data communication between the mobile device 500 and another computing device. The data port 514 can be a serial or a parallel port. In some instances, the data port 514 can be a USB port that includes data lines for data transfer and a supply line that can provide a charging current to charge the battery 530 of the mobile device 500.

The short-range communications subsystem 522 provides for communication between the mobile device 500 and different systems or devices, without the use of the wireless network 505. For example, the subsystem 522 may include an infrared device and associated circuits and components for short-range communication. Examples of short-range communication standards include standards developed by the Infrared Data Association (IrDA), Bluetooth, and the 802.11 family of standards developed by IEEE.

In use, a received signal such as a text message, an e-mail message, or web page download will be processed by the communication subsystem 504 and input to the main processor 502. The main processor 502 will then process the received signal for output to the display 510 or alternatively to the auxiliary I/O subsystem 512. A subscriber may also compose data items, such as e-mail messages, for example, using the keyboard 516 in conjunction with the display 510 and possibly the auxiliary I/O subsystem 512. The auxiliary subsystem 512 may include devices such as: a touch screen, mouse, track ball, infrared fingerprint detector, or a roller wheel with dynamic button pressing capability. The keyboard 516 is preferably an alphanumeric keyboard and/or telephone-type keypad. However, other types of keyboards may also be used. A composed item may be transmitted over the wireless network 505 through the communication subsystem 504.

For voice communications, the overall operation of the mobile device 500 is substantially similar, except that the received signals are output to the speaker 518, and signals for transmission are generated by the microphone 520. Alternative voice or audio I/O subsystems, such as a voice message recording subsystem, can also be implemented on the mobile device 500. Although voice or audio signal output is accomplished primarily through the speaker 518, the display 510 can also be used to provide additional information such as the identity of a calling party, duration of a voice call, or other voice call related information.

In particular, one of skill in the art will readily appreciate that the names of the methods and apparatus are not intended to limit implementations. Furthermore, additional methods and apparatus can be added to the components, functions can be rearranged among the components, and new components to correspond to future enhancements and physical devices used in implementations can be introduced without departing from the scope of implementations.

### CONCLUSION

The terminology used in this application is meant to include all THC or THC metabolite tests and alternate technologies which provide the same functionality as described herein.

1. An electronic device comprising:
a processor;
a display device; and
a memory operable to store processor instructions to:
receive at least one representation of characteristics of a person;
receive at least one representation of marijuana/cannabis use of the person;
determine a likelihood that the person will pass a drug test a certain period of time after cessation of intake of THC of the marijuana/cannabis, the likelihood represented as a percentage; and
display on the display device a visual presentation of the percentage of the likelihood.

2. The electronic device of claim 1, wherein the visual presentation further comprises:
a plot of a passing percentile (Y-axis) vs. Time (X-axis); and
a plot of six trendlines.

3. The electronic device of claim 2, wherein the plot of the six trendlines further comprises:
the plot of the six trendlines representing: Lab Tested Urine, Lab Tested Urine with Detox, Lab Tested Urine with Dilution, Home/On-Site Tested Urine, Home/On-Site Tested Urine with Detox, and Home/On-Site Tested Urine with Dilution.

4. The electronic device of claim 1, wherein the characteristics of the person further comprise:
gender, age, height, weight, waist size, body mass index (BMI), body fat percent, weight of body fat, estimated metabolic speed, cardio activity level, and muscular composition level.

5. The electronic device of claim 1, wherein the marijuana/cannabis use of the person further comprise:
smoking frequency, average marijuana potency, and average amount used.

6. The electronic device of claim 1, wherein the characteristics of the person further comprises:
THC or THC metabolite test results of the person.

7. The electronic device of claim 1, wherein the marijuana/cannabis use of the person consists of:
THC or THC metabolite test results of the person.

8. A non-transitory machine readable medium storing a program for enabling a computer to perform a method, comprising:
receiving representations of characteristics of a person;
receiving representations of marijuana/cannabis use of the person;
determining the likelihood that the person will pass a drug test a certain period of time after cessation of intake of THC, the likelihood represented as a percentage; and
displaying a visual presentation of the percentage of the likelihood.

9. The non-transitory machine readable medium of claim 8, wherein the visual presentation further comprises:
a plot of a passing percentile (Y-axis) vs. Time (X-axis); and
a plot of six trendlines.

10. The non-transitory machine readable medium of claim 9, wherein the plot of the six trendlines further comprises:
the plot of the six trendlines representing: Lab Tested Urine, Lab Tested Urine with Detox, Lab Tested Urine with Dilution, Home/On-Site Tested Urine, Home/On-Site Tested Urine with Detox, Home/On-Site Tested Urine with Dilution.

11. The non-transitory machine readable medium of claim 8, wherein the characteristics of the person further comprise:
gender, age, height, weight, waist size, body mass index (BMI), body fat percent, weight of body fat, estimated metabolic speed, cardio activity level, and muscular composition level.

12. The non-transitory machine readable medium of claim 8, wherein the marijuana/cannabis use of the person further comprise:
smoking frequency, average marijuana potency, and average amount used.

13. The non-transitory machine readable medium of claim 8, wherein the characteristics of the person further comprises:
THC or THC metabolite test results of the person.

14. The non-transitory machine readable medium of claim 8, wherein the marijuana/cannabis use of the person consists of:
THC or THC metabolite test results of the person.

15. A method comprising:
receiving representations of characteristics of a person;
receiving representations of the marijuana/cannabis use of the person;
determining a likelihood that the person will pass a drug test a certain period of time after cessation of intake of THC, the likelihood represented as a percentage; and
displaying a visual presentation of the percentage of the likelihood.

16. The method of claim 15, wherein the visual presentation further comprises:
a plot of a passing percentile (Y-axis) vs. Time (X-axis); and
a plot of six trendlines.

17. The method of claim 16, wherein the plot of the six trendlines further comprises:
the plot of the six trendlines representing: Lab Tested Urine, Lab Tested Urine with Detox, Lab Tested Urine with Dilution, Home/On-Site Tested Urine, Home/On-Site Tested Urine with Detox, Home/On-Site Tested Urine with Dilution.

18. The method of claim 15, wherein the characteristics of the person further comprise:
gender, age, height, weight, waist size, body mass index (BMI), body fat percent, weight of body fat, estimated metabolic speed, cardio activity level, and muscular composition level.

19. The method of claim 15, wherein the marijuana/cannabis use of the person further comprise:
smoking frequency, average marijuana potency, and average amount used.

20. The method of claim 15, wherein the characteristics of the person consists of:
THC or THC metabolite test results of the person.
